(19) United States
(12) Patent Application Publication
(10) Pub. No.: US 2023/0272120 A1
(43) Pub. Date: Aug. 31, 2023

(54) IMMUNOMAGNETIC COMPOSITIONS FOR THE PH-SPECIFIC CAPTURE OF EXTRACELLULAR VESICLES

(71) Applicant: The University of Kansas, Lawrence, KS (US)

(72) Inventor: Mei He, Gainesville, FL (US)

(21) Appl. No.: 17/998,177

(22) PCT Filed: May 10, 2021
(86) PCT No.: PCT/US2021/031590

§ 371 Date: Nov. 8, 2022

Related U.S. Application Data
(60) Provisional application No. 63/022,018, filed on May 8, 2020.

(51) Int. Cl. C07K 17/06; A61K 9/127; A61K 35/28

(52) U.S. Cl. CPC C07K 17/06; A61K 9/127; A61K 35/28

(57) ABSTRACT

The present disclosure provides immunomagnetic compositions and their methods of use, in particular magnetic particles conjugated to peptides that bind and capture extracellular vesicles.

### CROSS-REFERENCE TO RELATED APPLICATIONS

This application claims benefit of U.S. Provisional Application No. 63/022,018, filed May 8, 2020, which is hereby incorporated herein by reference in its entirety.

### STATEMENT REGARDING FEDERALLY SPONSORED RESEARCH OR DEVELOPMENT

This invention was made with government support under GM133794 awarded by the National Institutes of Health. The government has certain rights in the invention.

### TECHNICAL FIELD

This disclosure relates to immunomagnetic compositions, and more particularly to compositions comprising magnetic particles bound to a pH responsive peptide that can reversibly bind to extracellular vesicles to allow capture and release of the extracellular vesicles with adjustment of pH.

### BACKGROUND

Extracellular vesicles (EVs) and exosomes are live cell secreted small membrane vesicles. Extracellular vesicle (EV) is a loose term, which typically describes three types of vesicles: exosomes, microvesicles, and apoptotic bodies. The major differences between these three vesicles are their cellular origins and molecular pathways. The formation of exosomes begins with the creation of endosomes as intracellular vesicles, then followed with multi-vesicular body (MVB) fusion with the plasma membrane and secreted its contents into the extracellular space. Exosomes are in size between 30-150 nm. EVs and exosomes are derived from living cells, including mammalian cells, bacteria (named as outer membrane vesicles), fungi and eukaryotic parasites. Such small vesicles are highly biocompatible and enclose specific subsets of proteins, DNAs, RNAs and lipids capable of stimulating antigen-specific immune response. EVs and exosomes contain a lipid bilayer and protein content derived from their parent cells. EVs and exosomes have been observed to play a vital role in communication, delivery, and mediation of diseases, without the need of cell-cell contact. Upon their release from the parent cell, EVs can either bind to local cells, the extracellular matrix, cross biological barrier, or enter bodily fluids such as blood or cerebrospinal fluid. Such movement allows EVs to deliver important contents and signals to cells both locally and distant. Nowadays, exosomes and EVs have been recognized as the most emerging delivery and therapeutic agents for cancer immunotherapy, regenerative medicine, and precision drug delivery. Unfortunately, there are no good solutions to precisely isolate and purify exosome populations. Current exosomes are pooled from a large population of cells, and the understanding of exosome biology completely stems from these ensemble-average measurements of exosome properties. There is a clear need for compositions and methods that may be used to isolate and optionally modify extracellular vesicles.

### SUMMARY

The present disclosure provides immunomagnetic compositions that are capable of binding to extracellular vesicles and facilitate their isolation using a magnetic field, where the immunomagnetic composition binds and releases the extracellular vesicles in a pH dependent manner. This allows for a more general binding and capture of extracellular vesicles within any medium (such as a biological medium) without requiring binding to specific membrane components present in the extracellular vesicles, such as membrane proteins. Further, the immunomagnetic compositions bind to the extracellular vesicles by use of a pH responsive peptide capable of binding or release within the lipid bilayer of the extracellular vesicle depending upon the pH of the surrounding medium and which can be recycled for multiple uses.

Thus, in one aspect, an immunomagnetic composition is provided comprising a population of magnetic particles, wherein each magnetic particle is conjugated to at least one pH responsive extracellular vesicle-binding peptide.

In some embodiments, the magnetic particle can be ferromagnetic, paramagnetic, or super-paramagnetic. In some embodiments, the population of magnetic particles has an average particle size ranging from about 1 nm to about 100 microns, for example from about 1 micron to about 50 microns, from about 1 micron to about 10 microns, or from about 1 micron to about 3 microns. In some embodiments, the magnetic particle comprises a magnetic element, for example iron, nickel, and cobalt, or oxide compounds thereof. In some embodiments, the magnetic particle has a surface. In some embodiments, a graphene oxide nanomaterial is covalently bound to the surface. In some embodiments, polydopamine is further covalently bound to the graphene oxide nanomaterial.

In some embodiments, the peptide is conjugated to the surface. In some embodiments, the peptide is conjugated to the polydopamine. In some embodiments, the peptide is covalently bound to the surface. In some embodiments, the peptide is covalently bound to the polydopamine. In some embodiments, the peptide is conjugated to the surface or the polydopamine by a covalent linker. In some embodiments, the surface or the polydopamine comprises one or more streptavidin groups. In some embodiments, the peptide comprises a biotinylated residue, wherein the biotinylated residue is bound to the streptavidin group.

In some embodiments, the peptide comprises an amino acid sequence having at least 80% identity (for example 85%, 90%, 95%, or more identity) with an amino acid sequence selected from SEQ ID NO: 1 to 21. In some embodiments, the peptide consists of an amino acid sequence having at least 80% identity (for example 85%, 90%, 95%, or more identity) with an amino acid sequence selected from SEQ ID NO: 1 to 21. In some embodiments, the peptide has an amino acid sequence selected from SEQ ID NO: 1 to 21.

Further provided is a method for isolating a population of extracellular vesicles from a medium, for example a biological fluid, the method comprising:
contacting the medium with an immunomagnetic composition as described herein and optionally an aqueous solution to form a mixture;
adjusting the pH of the mixture to within a range from about 3 to about 5, for example from about 3.5 to about 4.5, to bind the population of magnetic particles to the population of extracellular vesicles; and
collecting the population of extracellular vesicles bound to the magnetic particles.

In some embodiments, the extracellular vesicles may comprise ectosomes, microvesicles (MV), microparticles (MP), exosomes, apoptotic bodies, large oncosomes, exophers, platelets, red blood cells, enveloped viruses, and exomeres. In particular embodiments, the extracellular vesicles comprise exosomes.

In some embodiments, the medium comprises a biological fluid, for example blood (such as arterial or venous blood), cerebrospinal fluid, peritoneal fluid, pleural fluid, amniotic fluid, or lymphatic fluid.

In some embodiments, the method may further comprise contacting the population of extracellular vesicles bound to the population of magnetic particles with an agent such that the agent is encapsulated within the extracellular vesicles. In some embodiments, the agent may be a therapeutic agent, for example a drug. In some embodiments, the agent comprises an anti-cancer agent. In other embodiments, the agent may comprise a vaccine, an immunotherapy agent, or a regenerative therapy agent. In some embodiments, the agent may comprise a peptide, an siRNA, a microRNA, one or more components of a Crispr-Cas9 system, a plasmid, or an enzyme.

In some embodiments, the method may further comprise contacting the population of extracellular vesicles bound to the population of magnetic particles with a surface modifying agent. In some embodiments, each extracellular vesicle comprises a lipid bilayer, and the surface modifying agent inserts within the lipid bilayer. In some embodiments, the surface modifying agent chemically modifies a component of the lipid bilayer, for example a membrane lipid or protein. In some embodiments, the surface modifying agent may comprise a tissue penetration peptide, a tissue homing peptide, an immunity activating agent, or a receptor activating agent.

In some embodiments, the method may further comprise releasing the population of extracellular vesicles from the magnetic particles by adjusting the pH within a pH range of about 7 to about 9, for example from about 7.5 to about 8.5.

The present disclosure also provides a population of extracellular vesicles prepared by any of the methods described herein.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A is a schematic depicting immunomagnetic insertion peptide beads for pH-controlled, on demand capture and release of extracellular vesicles and exosomes.

FIG. 1B is a schematic depicting a method of using immunomagnetic insertion peptide beads for 1) extracellular vesicle and exosome isolation, 2) cargo internal loading, and 3) surface engineering and decoration of extracellular vesicles and exosomes, and lastly release of captured extracellular vesicles and exosomes on demand.

FIG. 2 is schematic illustration of inhalable mesenchymal stem cell secreted exosomes (MSC-Exos) with insertion peptide modifications.

Like reference symbols in the various drawings indicate like elements.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptation to the present disclosure are possible and can even be desirable in certain circumstances and are a part of the present disclosure. Thus, the following description is provided as illustrative of the principles of the present disclosure and not in limitation thereof.

### Definitions

Unless defined otherwise, all technical and scientific terms used herein have the same meaning as commonly understood to one of ordinary skill in the art to which the invention belongs. The following definitions are provided for the full understanding of terms used in the specification.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a particular magnetic particle is disclosed and discussed and a number of modifications that can be made to the magnetic particle are discussed, specifically contemplated is each and every combination and permutation of the magnetic particles and the modifications that are possible unless specifically indicated to the contrary. Thus, if a class of magnetic particles A, B, and C are disclosed as well as a class of magnetic particles D, E, and F and an example of a combination magnetic particle, or for example, a combination magnetic particle comprising A-D is disclosed, then even if each is not individually recited each is individually and collectively contemplated meaning combinations A-E, A-F, B-D, B-E, B-F, C-D, C-E, and C-F are considered disclosed. Likewise, any subset or combination of these is also disclosed. Thus, for example, the sub-group A-E, B-F, and C-E would be considered disclosed. This concept applies to all aspects of this disclosure including, but not limited to, steps in methods of making and using the disclosed compositions. Thus, if there are a variety of additional steps that can be performed, it is understood that each of these additional steps can be performed with any specific embodiment or combination of the disclosed methods.

It is understood that the composition disclosed herein have certain functions. Disclosed are certain structural requirements for performing the disclosed functions, and it is understood that there are a variety of structures which can perform the same function which are related to the disclosed structures, and these structures will ultimately achieve the same results.

Unless otherwise expressly stated, it is in no way intended that any method set forth herein be constructed as requiring that its steps be performed in a specific order. Thus, where a method claim does not expressly recite an order of steps to be followed or it is not otherwise specifically stated in the claims or description that the steps are to be limited to a specific order, it is no way intended that an order be inferred, in any respect. This holds for any possible non-express basis for interpretation, including matter of logic with respect to arrangement of steps or operation flow; plain meaning derived from grammatical organization or punctuation; and the number or type of embodiments described in the specification.

As used in the specification and claims, the singular form “a”, “an”, and “the” include plural referents unless the context clearly dictates otherwise. For example, the term “a particle” includes a plurality of particles, including mixtures thereof.

As used herein, the terms “can”, “may”, “optionally”, “can optionally”, and “may optionally” are used interchangeably and are meant to include cases in which the condition occurs as well as cases in which the condition does not occur. Thus, for example, the statement that a composition “may include an excipient” is meant to include cases in which the composition includes an excipient as well as cases in which the formulation does not include an excipient. Ranges can be expressed as from “about” one particular value, and/or to “about” another particular value. When such a range is expressed, another embodiment, includes from the one particular value and/or to the other particular value. Similarly, when values are expressed as approximations, by use of the antecedent “about”, it will be understood that the particular value forms another embodiment. It is also understood that the endpoints of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

“Amino acid” as used herein refers to a molecule containing both an amino group and a carboxyl group. Amino acids include α-amino acids and β-amino acids. In certain forms, an amino acid is an alpha amino acid. Amino acids can be natural or synthetic. Amino acids include, but are not limited to, the twenty standard or canonical amino acids: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trk, W), Tyrosine (Tyr, Y), and Valine (Val, V). Common non-standard or non-canonical amino acids include, but are not limited to, selenocysteine, pyrrolysine, and N-formylmethionine. The term “synthetic amino acid” or “non-natural amino acid” as used herein refers to an organic compound that a structure similar to a natural amino acid so that it mimics the structure and reactivity of a natural amino acid. The synthetic amino acid as defined herein generally increases or enhances the properties of a peptide (e.g., selectivity or stability) when the synthetic amino acid is either substituted for a natural amino acid or incorporated into a peptide.

The terms “peptide”, “protein”, “polypeptide”, or “polyamino acid” are used interchangeably to refer to a natural or synthetic molecule comprising two or more amino acids linked by the carboxyl group of one amino acid to the amino group of another. In addition, as used herein, the term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translation processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in the given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleoside or nucleotide derivative, covalent attachment of a lipid or lipid derivative, covalent attachment of a phosphatidylinositol, disulfide bond formation, demethylation, formation of cysteine or pyroglutamate, formylation, gamma-carboxylation, glycosylation, GPI anchor formation, hydroxylating, iodination, methylation, myristoylation, oxidation, PEGylation, proteolytic processing, phosphorylation, prenylation, racemization, selenoylation, sulfation, and transfer-RNA mediated addition of amino acids to proteins such as arginylation. Also included in the term “polypeptides” are cis- and trans-isomers, R- and S-enantiomers, D-isomers, L-isomers, diastereomers, conformers, and mixtures thereof.

The term “residue” as used herein refers to an amino acid that is incorporated into a polypeptide. The amino acid may be a naturally occurring amino acid and, unless otherwise limited, may encompass analogs of natural amino acids that can function in a similar manner as naturally occurring amino acids.

A “variant” as used herein means a polypeptide comprising one or more modifications such as substitutions, deletions, and/or truncations of one or more specific amino acid residues in the corresponding wild-type peptide. A variant of a polypeptide may be naturally occurring or synthetic, and may have 50%, 55%, 60%, 65%, 70%, 75%, 80%, 85%, 90%, 95%, 96%, 97%, 98% or 99% identity with the wild-type polypeptide.

The term “identical” or percent “identity”, in the context of two or more peptide sequences, refer to two or more sequences or subsequences that are the same or have a specified percentage of amino acid residues that are the same (i.e., about 60% identity, preferably 61%, 62%, 63%, 64%, 65%, 66%, 67%, 68%, 69%, 70% 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical”. This definition includes sequences that have deletions and/or additions, as well as those that have substitutions. As described below, the preferred algorithms can account for gaps and the like. Preferably, identity exists over a region that is at least about 10 amino acids in length, or more preferably over a region that is 10-50 amino acids in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieve in various ways that are within the skill in the art, for instance, using publicly available computer software such as BLAST, BLAST-2, ALIGN, ALIGN-2, or Megalign software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402 and Altschul et al. (1990) J. Mol. Bio. 215:403-410, respectively. Software for performing BLAST analyses is publicly available through the National Center for Biotechnology Information (http://www.ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (199) J. Mol. Bio. 215:403-410). These initial neighborhood word hits act as seeds for initiating searches to find longer HSPs containing them. The word hits are extended in both directions along each sequence for as far as the cumulative alignment score can be increased. For amino acid sequences, a scoring matrix is used to calculate cumulative score. Extension fo the word hits in each direction are halted when the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. For amino acid sequences, the BLASTP program uses as defaults a word length of 3, and expectation (E) of 10, and the BLOSUM62 scoring matrix (see Henikoss and Henikoss (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The present disclosure provides immunomagnetic compositions that may be used for the capture of extracellular vesicles from biological fluids, methods of using the disclosed immunomagnetic compositions, as well as populations of extracellular vesicles isolated and/or modified using these disclosed methods. The present immunomagnetic compositions selectively bind to extracellular vesicles at a pH from about 3 to about 5, allowing capture of the extracellular vesicles by application of a magnetic field. Once isolated, the extracellular vesicles can be released from the immunomagnetic composition

Thus, the present disclosure provides immunomagnetic compositions comprising a population of magnetic particles, wherein each magnetic particle is conjugated to a pH responsive extracellular vesicle-binding peptide.

Magnetic Particles

In one aspect, the immunomagnetic compositions of the present disclosure comprise magnetic particles. The magnetic particles may be of any shape, including but not limited to spherical, rod, elliptical, cylindrical, and disc. In some embodiments, magnetic particles having a substantially spherical shape and defined surface chemistry can be used to minimize chemical agglutination and non-specific binding. As used herein, the term “magnetic particles” can refer to a nano- and micro-scale particle that is attracted or repelled by a magnetic field gradient or has a non-zero magnetic susceptibility. The magnetic particles can be ferromagnetic, paramagnetic, or super-paramagnetic. In some embodiments, the magnetic particles can be super-paramagnetic.

The population of magnetic particles can have an average particle size ranging from about 1 nm to about 100 micron. In some embodiments, the population of magnetic particles can have an average particle size ranging from about 1 micron to 100 microns, from 1 micro to 50 microns, from 1 micron to 20 microns, or from 1 micron to 10 microns. In some embodiments, the population of magnetic particles can have an average particle size from about 500 nm to about 5 microns, from about 1 micron to about 5 microns, or from about 1 micron to about 3 microns. Magnetic particles are a class of particles which can be manipulated using a magnetic field and/or a magnetic field gradient. Such particles commonly consist of magnetic elements such as iron, nickel, or cobalt, or oxide compounds thereof. Magnetic particles (including nanoparticles or microparticles) are well-known and methods for their preparation have been described in the art. Magnetic particles are also widely and commercially available.

In some embodiments, the magnetic particle comprises a surface and further comprises a graphene oxide layer on the surface. In some embodiments, the magnetic particle comprises at least one polydopamine polymer coupled to the graphene oxide layer. In some embodiments, the graphene oxide layer is covalently coupled to the surface. In some embodiments, the at least one polydopamine polymer is covalently coupled to the graphene oxide layer. In such embodiments, the magnetic particle comprises a graphene oxide-polydopamine nanomaterial bound to its surface. The graphene oxide is linked to the surface of the magnetic particle and has polydopamine linked thereof, where the polydopamine includes a peptide as described herein that can bind to an extracellular vesicle, for example an exosome. In some embodiments, the polydopamine can comprise pores, such as micro-pores or nano-pores, which provide a unique morphology for capturing the extracellular vesicle targets as described herein.

In some embodiments, graphene oxide-polydopamine coating can be used to modify the surfaces of magnetic particles, such as magnetic beads. While workable, a non-covalently assembled nano-graphene coating can suffer from instability in buffer solutions over time. As such, covalent bonding may be advantageous. In a typical embodiment, a method for forming a graphene oxide-polydopamine magnetic bead can be provided. In the present method, encapsulated Fe₃O₄/SiO₂ core-shell nanoparticles can be coated with grapen-oxide nanosheets via carboxamide covalent bonds formed by EDC/NHS chemistry and modified with (3-aminopropyl)triethoxysilane (APTES) which leads to substantially improved stability. APTES results in amine groups on the nanoparticle that can react with the carboxylic acid groups of the graphene oxide. Typically, nanographene coated magnetic particles show much larger surface area than their uncoated counterparts. Upon coating with polydopamine, the magnetic particles are found to have a porous surface structure which indicated enhanced specific surface area allowing for immobilization of an increased quantity of the peptides described herein. The polydopamine layer is deposited on the graphene oxide surface by reacting amino groups of the polydopamine with carboxylic acid groups of the graphene oxide such that the polydopamine polymers extend from the graphene oxide surface away from the particle core. The polydopamine conjugated to the graphene oxide still retains amino groups that can be used to react with carboxylic acid groups, such as the terminal carboxylic acid, found in the peptides described herein. This results in the polydopamine extending the peptide away from the particle core. The deposition of polydopamine layers creates a 3D hydrophilic, nanostructured interface to enhance the affinity capture of the targeted extracellular vesicles.

With regard to the polydopamine, the amine and catechol functional groups found therein allow easy surface modification and bioconjugation of the peptides described herein. The highly hydrophilic polydopamine coating possesses excellent biocompatibility and resistance to biofouling. The kinetics of polydopamine coating can be well controlled by tuning the reaction condition such as pH, temperature, choice of oxidants and incubation time. The bead surface functionalized with a graphene oxide-induced, nanostructured polydopamine film by microfluidic layer-by-layer coating permits simple covalent conjugation of the peptides described herein via the chemistry of the polydopamine. In one aspect, the coating approach markedly expedites the polydopamine deposition kinetic and can be complete within one hour, which could promote greater application of this promising coating material.

In some embodiments, the magnetic particles can be coated with one member of an affinity binding pair that can facilitate the conjugation of the magnetic particles to the peptides described herein for capturing extracellular vesicles. The term “affinity binding pair” or “binding pair” refers to first and second molecules that specifically bind go teach other. One member of the binding pair is conjugated with the first part to be linked (e.g., the magnetic particles) while the second member is conjugated with the second part to be linked (e.g., the peptides described herein). Exemplary binding pairs include any haptenic or antigenic compound in combination with a corresponding antibody or binding portion or fragment thereof (e.g., digoxigenin and antidigoxigenin; mouse immunoglobulin and goat antimouse immunoglobulin) and nonimmunological pairs (e.g., biotin-avidin, biotin-streptavidin, biotin-neutravidin, hormone [e.g., thyroxine and cortisol-hormone binding protein, receptor-receptor agonist, receptor-receptor antagonist (e.g., acetylcholine receptor-acetylcholine or an analog thereof), IgG-protein A, IgG-protein G, IgG-synthesized protein AG, lectin-carbohydrate, enzyme-enzyme cofactor, enzyme-enzyme inhibitor, and complementary oligonucleotide pairs capable of forming nucleic acid duplexes), and the like. The binding pair can also include a first molecule which is negatively charged and a second molecule which is positively charged.

One example of using binding pair conjugation is the biotin-avidin, biotin-streptavidin or biotin-neutravidin conjugation. Accordingly, in some embodiments, the magnetic particles can be coated with avidin-like molecules (e.g., streptavidin or neutravidin), which can be conjugated to biotinylated linkages to the peptides described herein.

Peptides

In another aspect, the present disclosure provides pH responsive extracellular vesicle-binding peptides that are conjugated to the magnetic particles of the immunomagnetic compositions described herein. Such peptides are able to bind to extracellular vesicles by intercalating within the lipid bilayer of said vesicles. Upon adjustment of the environmental pH within a specific range, the peptide folds within a specific conformation that leads to stronger binding within the lipid bilayer, leading to formation of a complex of the immunomagnetic composition and the extracellular vesicles. Upon isolation of this complex, the extracellular vesicles can be subsequently released by adjusting the environmental pH out of the above-recited range.

The behavior of the peptides described herein typically involves consisting in three states. At physiological pH, the peptides exist in an equilibrium between a solvated state and a membrane-adsorbed state; a decrease in pH then shifts the equilibrium to a membrane-inserted state.

In some embodiments, the peptide comprises a hydrophobic middle region and a C-terminal membrane-inserting region, wherein one or more protonatable residues are interspersed throughout each region. At physiological pH, the one or more protonatable residues are negatively charged. Upon a decrease in pH, the one or more protonatable residues become neutral. The loss of charge and increase in overall hydrophobicity drives the peptides described herein to partition across the hydrophobic core of the membrane bilayer to form a transmembrane helix. This helix spans the lipid bilayer, leaving the N-terminus outside of the extracellular vesicle and the C-terminus within the extracellular vesicle. The C-terminus can also again become deprotonated and charged due to the more alkaline pH found in such an environment, an effect that stably anchors the peptide within the membrane.

The peptide can be a synthetic peptide containing non-natural amino acids, or a peptidomimetic. As used herein, “peptidomimetic” means a mimetic of a peptide which includes some alteration of the normal peptide chemistry. Peptidomimetics typically enhance some property of the original peptide, such as increased stability, increased efficacy, enhanced delivery, increased half-life, etc. Use of peptidomimetics can involve the incorporation of a non-amino acid residue with non-amide linkages at a given position. One embodiment of the present invention is a peptidomimetic wherein the compound has a bond, a peptide backbone, or an amino acid component replaced with a suitable mimic. Some non-limiting examples of non-natural amino acids which may be suitable amino acid mimics include, but are not limited to, β-alanine, L-α-aminobutyric acid, L-γ-aminobutyric acid, L-α-aminoisobutyric acid, L-ε-aminocaproic acid, 7-aminoheptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δ-Cbz-L-ornithine, N-δ-Boc-N-α-Cbz-L-orinithine, Boc-p-nitro-L-phenylalanine, Boc-hydroxyproline, and Boc-L-thioproline.

The disclosed peptides may also be substituted with any number of substituents or functional moieties. In general, the term “substituted” refers to the replacement of a hydrogen group in a given structure with a specified substituent group. When more than one position in any given structure may be substituted with more than one substituent selected from a specified group, the substituent may be either the same or different at every position. As used herein, the term “substituted” is contemplated to include substitution with all permissible substituents of organic compounds, any of the substituents described herein (for example aliphatic, alkyl, alkenyl, alkynyl, heteroaliphatic, heterocyclic, aryl, heteroaryl, acyl, oxo, imino, thioxo, cyano, isocyano, amino, azido, nitro, hydroxyl, thio, halo, etc.), and any combination thereof (for example, aliphatic amino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like) that results in the formation of a stable moiety. The disclosed peptides can contain any and all such combinations in order to arrive at a stable substituent/moiety. For the disclosed peptides, heteroatoms such as nitrogen may have hydrogen substituents and/or any suitable substituent as described herein which satisfy the valencies of the heteroatoms and results in the formation of a stable moiety.

Peptides and peptidomimetics can be prepared by any method, such as by synthesizing the peptide or peptidomimetic, or by expressing a nucleic acid encoding an appropriate amino acid sequence in a cell and harvesting the peptide from the cell. Of course, a combination of such methods also can be used.

Examples of chemical synthesis technologies are solid phase synthesis and liquid phase synthesis. Solid phase synthesis methods are largely classified by the tBoc method and the Fmoc method, depending on the type of protective group used. Typically used protective groups include tBoc (t-butoxycarbonyl), Cl—Z (2-chlorobenzyloxycarbonl), Br—Z (2-bromobenzyloxycarbonyl), Bzl (benzyl), Fmoc (9-fluorenylmethoxycarbonyl), Mbh (4,4′-dimethoxydibenzyhydryl), Mtr (4-methoxy-2,3,6-trimethylbenzenesulfonyl), Trt (trityl), Tos (tosyl), Z (Benzyloxycarbonyl), and Clz-Bzl (2,6-dichlrobenzyl) for the amino groups; NO2 (nitro) and Pmc (2,2,5,7,8-pentamethylchromane-6-sulfonyl) for the guanidino groups; and t-Bu (t-butyl) for the hydroxyl groups. After synthesis of the desired peptide, it is subjected to one or more deprotection reactions and cut out from the solid support. Such peptide cutting reactions may be carried out with hydrogen fluoride or trifluoromethane sulfonic acid for the Boc method, or with TFA for the Fmoc method. Methods of de novo synthesizing of peptides and peptidomimetics are described, for example, in Chan et al., Fmoc Solid Phase Peptide Synthesis, Oxford University Press, Oxford, United Kingdom, 2005; and Peptide and Protein Drug Analysis, ed. Redi., R., Marcel Dekker, Inc., 2000.

Alternatively, the peptide may be synthesized using recombinant techniques. In this case, a nucleic acid encoding the peptide is cloned into an expression vector under the control of expression control sequences (e.g., a promoter, a terminator and/or an enhancer) allowing its expression. The expression vector is then transfected into a host cell (e.g., a human, CHO, mouse, monkey, fungal or bacterial host cell), and the transfected host cell is cultivated under conditions suitable for the expression of the peptide. Standard recombinant DNA and molecular cloning techniques are described, for example, in: Sambrook and Maniatis, Molecular Cloning: A Laboratory Manual, Second Edition, Cold Spring Harbor Laboratory Press, Cold Spring Harbor, N.Y. (1989); Silhavy et al., Experiments with Gene Fusions, Cold Spring Harbor Laboratory Press, Cold Spring Harbor, N.Y. (1984); and Ausubel et al., Current Protocols in Molecular Biology, published by Greene Publishing Assoc. and Wiley-Interscience (1987).

The method of producing the peptide may optionally comprise the steps of purifying said peptide, chemically modifying said peptide, and/or formulating said peptide into a pharmaceutical composition.

In some embodiments, the peptide comprises an amino acid sequence with at least 80% sequence identity with at least one of the amino acid sequences provided in the table below:

SEQ ID NO.: Amino Acid Sequence
1 AEQNPIYWARYADWLFTTPLLLLDLALLVDADEGT
2 AEQNPIYWARYADWLFTTALLLLDLALLVDADEGT
3 AEQNPIYWARYAGlaWLFTTPLLL1DLALLVDADEGT
4 AEDNPIYWARYAGlaWLFTTPLLLAadLALLVDADEGT
5 ADDQDPWRAYLDLLFPTDTLLLDLLW
6 ADDQNPWRAYLGlaLLFPTDTLLLDLLW
7 GEEQNPWLGAYLDLLFPLELLGLLELGLWG
8 GLAGLAGLLGLEGLLGLPLGLLELLWLGLELEGN
9 GGEQNPIYWARYADWLFTTPLLLLDLALLVDADEGT
10 ACEQNPIYWARYADWLFTTPLLLLDLALLVDADEGT
11 Ac-AKEQNPIYWARYADWLFTTPLLLLDLALLVDADECT
12 Ac-AKEQNPIYWARYAGlaWLFTTPLLLLDLALLVDADECT
13 Ac-AKEQNPIYWARYAGlaWLFTTPLLLLAadLALLVDADECT
14 ACDDQNPWRAYLDLLFPTDTLLLDLLWA
15 ADDQNPWRAYLDLLFPTDTLLLDLLWCA
16 ACDDQNPWRAYLGlaLFPTDTLLLDLLWG
17 ADDQNPWRAYLGlaLLFPTDTLLLDLLWCG
18 Ac-GKEEQNPWLGAYLDLLFPLELLGLLELGLWCG
19 ACGLAGLAGLLGLEGLLGLPLGLLEGLWLGLELEGN
20 GLAGLAGLLGLEGLLGLPLGLLEGLWLGLELEGNCA
21 ACDDQNPWRAYLDLLFPTDTLLLDLLW
wherein Gla is γ-carboxyglutamic acid and Aad is α-aminoadipic acid.

In some embodiments, the peptide comprises an amino acid sequence with at least 85% sequence identity with an amino acid sequence selected from SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID to NO: 18, SEQ. ID NO: 19, SEQ. ID NO: 20, or SEQ. ID NO: 21. In some embodiments, the peptide comprises an amino acid sequence with at least 90% sequence identity with an amino acid sequence selected from SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, or SEQ. ID NO: 21. In some embodiments, the peptide comprises an amino acid sequence with at least 95% sequence identity with an amino acid sequence selected from SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, or SEQ. ID NO: 21.

In some embodiments, the peptide consists of an amino acid sequence with at least 80% sequence identity with an amino acid sequence selected from SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, or SEQ. ID NO: 21. In some embodiments, the peptide consists of an amino acid sequence with at least 85% sequence identity with an amino acid sequence selected from SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, or SEQ. ID NO: 21. In some embodiments, the peptide consists of an amino acid sequence with at least 90% sequence identity with an amino acid sequence selected from SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, or SEQ. ID NO: 21. In some embodiments, the peptide consists of an amino acid sequence with at least 95% sequence identity with an amino acid sequence selected from SEQ. ID NO: 1, SEQ. ID NO: 2, SEQ. ID NO: 3, SEQ. ID NO: 4, SEQ. ID NO: 5, SEQ. ID NO: 6, SEQ. ID NO: 7, SEQ. ID NO: 8, SEQ. ID NO: 9, SEQ. ID NO: 10, SEQ. ID NO: 11, SEQ. ID NO: 12, SEQ. ID NO: 13, SEQ. ID NO: 14, SEQ. ID NO: 15, SEQ. ID NO: 16, SEQ. ID NO: 17, SEQ. ID NO: 18, SEQ. ID NO: 19, SEQ. ID NO: 20, or SEQ. ID NO: 21.

Addition of water-soluble polymers or carbohydrates to peptide drugs has been shown to prevent their degradation and increase their half-life. For instance, “PEGylation” of polypeptide drugs protects them and improves their pharmacodynamic and pharmacokinetic profiles. The PEGylation process attaches repeating units of polyethylene glycol (PEG) to a polypeptide drug. PEGylation of molecules can lead to increased resistance of drugs to enzymatic degradation, increase half-life in vivo, reduced dosing frequency, decreased immunogenicity, increased physical and thermal stability, increased solubility, increase liquid stability, and reduced aggregation. Therefore, in some embodiments the disclosed polypeptide is covalently linked to a water soluble polymer, such as polyethylene glycol.

The most common route for PEG conjugation of polypeptides has been to activate the PEG with functional groups suitable for reactions with lysine and N-terminal amino acid groups. The monofunctionality of methoxyPET makes it particularly suitable for protein and peptide modification because it yields reactive PEGs that do not produce cross-linked polypeptides, as long as diol PEG has been removed. Branched structures of PEG have also been proven to be useful for PEGylation of a protein or a peptide. For example, a branched PEG attached to a protein has properties of a much larger molecule than a corresponding linear mPEG of the same molecular weight. Branched PEGS also have the advantage of adding two PEG chains per attachment site on the protein, therefore reducing the chance of protein inactivation due to attachment. Furthermore, these structures are more effective in protecting proteins from proteolysis, in reducing antigenicity, and in reducing immunogenicity.

The peptides as used in the immunomagnetic compositions described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups, can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), Sigma (St. Louis, Mo.), Pfizer (New York, N.Y.), GlaxoSmithKline (Raleigh, N.C.), Merck (Whitehouse Station, N.J.), Johnson & Johnson (New Brunswick, N.J.), Aventis (Bridgewater, N.J.), AstraZeneca (Wilmington, Del.), Novartis (Basel, Switzerland), Wyeth (Madison, N.J.), Bristol-Myers-Squibb (New York, N.Y.), Roche (Basel, Switzerland), Lilly (Indianapolis, Ind.), Abbott (Abbott Park, Ill.), Schering Plough (Kenilworth, N.J.), or Boehringer Ingelheim (Ingelheim, Germany), or are prepared by methods known to those skilled in the art following procedures set forth in the references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry (John Wiley and Sons, 4^{th} Edition); and Larock's Comprehensive Organic Transformations (VCH Publishers Inc., 1989). Other materials, such as the pharmaceutical carriers disclosed herein, can be obtained from commercial sources.

Reactions to produce the compounds described herein can be carried out in solvents, which can be selected by one of skill in the art of organic synthesis. Solvents can be substantially reactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formulation can be monitored by spectroscopic means, such as nuclear magnetic resonance spectroscopy (e.g., ¹H or ¹³C), infrared spectroscopy, spectrophotometry (e.g. UV-visible), or mass spectrometry, or by chromatography such as high-performance liquid chromatography (HPLC) or thin layer chromatography.

The disclosed compounds can be prepared by solid phase peptide synthesis wherein the amino acid α-N-terminal is protected by an acid or base protecting group. Such protecting groups should have the properties of being stable to the conditions of peptide linkage formation while being readily removable without destruction of the growing peptide chin or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are: for side chain amino groups like lysine and arginine, 2,2,5,7,8-pentamethylchroman-6-sunfonyl (pmc), nitro, p-toluenesulfonyl, 4-methoxybenzene-sulfonyl, Cbz, Boc, and adamantyloxycarbonyl; for tyrosine, benzyl, o-bromobenzyloxycarbonyl, 2,6-dichlorobenzyl, isopropyl, t-butyl (t-Bu), cyclohexyl, cyclopentyl, and acetyl (Ac); for serine, t-butyl, benzyl, and tetrahydropyranyl; for histidine, trityl, benzyl, Cbz, p-toluenesulfonyl and 2,4-dinitrophenyl; for tryptophan, formyl; for aspartic and glutamic acid, benzyl and t-butyl; and for cysteine, triphenylmethyl (trityl). In solid phase peptide synthesis methods, the α-C-terminal amino acid is attached to a suitable solid support or resin. Suitable solid supports useful for the above synthesis are those materials which are inert to the reagents and reaction of the stepwise condensation-deprotection reactions, as well as being insoluble in the media use. Solid supports for synthesis of α-C-terminal carboxy peptides include 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif.). N,N′-diisopropylcarbodiimide (DIC) or O-benzotriazol-1-yl-N,N,N′N′-tetramethyluronium hexafluorophosphate (HBTU), with or without 4-dimethylaminopyridine (DMAP), 1-hydroxybenzotriazole (HOBt), benzotriazole-1-yloxy-tris(dimethylamino)phosphonium hexafluorophosphate (BOP) or bis(2-oxo-3-oxazolidinyl)phosphine chloride (BOPC1), mediate coupling for from about 0.5 to about 24 hours at a temperature of between 10° C. and 50° C. in a solvent such as dichloromethane, DMF, or NMP. When the solid support is 4-(2′,4′-dimethoxyphenyl-Fmoc-aminomethyl)phenoxy-acetamidoethyl resin, the Fmoc group is cleaved with a secondary amine, preferably piperidine, prior to coupling with the α-C-terminal amino acid as described above. One method for coupling to the deprotected (3′,4′-dimethoxyphenyl-Fmoc-aminomethyl)phenoxy-acetamidoethyl resin is O-benzotriazol-1-yl-N,N,N′,N′-tetramethyluronium hexafluorophosphate (HBTU, 1 equiv.) and 1-hydroxybenzotriazole (HOBt, 1 equiv.) in DMF or O-(1H-6-chlorobenzotriazol-1-yl)-1,1,3,3,-tetramethyluronium hexafluorophosphate (HCTU, 1 equiv.) and N,N-diisopropylethylamine (DIEA, 1 equiv.) in NMP. The coupling of successive protected amino acids can be carried out in an automatic polypeptide synthesize. In one example, the α-N-terminal in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N′,N′-tetramethyluronium hexafluorophosphate (HBTU, 1 equiv.) and 1-hydroxybenzotriazole (HOBt, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleave reagent comprising thianisole, water, ethanedithiol, and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography or underivatized polystyrene-divinylbenzene (for example, Amberlite XED); silica gel adsorption chromatography; ion exchange chromatography on carboxymethylcellulose; partition chromatography, e.g. on Sephadex G-25, LH-20 or countercurrent distribution; or high performance liquid chromatography (HPLC), especially reverse-phase HPLC on octyl- or octadecylsilyl-silica bonded column packing.

### Method of Use

The present disclosure also provides methods for isolating a population of extracellular vesicles by using the immunomagnetic compositions described herein.

Thus in one aspect, a method for isolating a population of extracellular vesicles from a medium, for example a biological fluid is provided comprising:
contacting the medium with an immunomagnetic composition as described herein and optionally an aqueous solution to form a mixture;
adjusting the pH of the mixture to within a range of about 3 to about 5 to bind the population of magnetic particles to the population of extracellular vesicles; and
collecting the population of extracellular vesicles bound to the magnetic particles by applying a magnetic field.

“Extracellular vesicles” are lipid bilayer-delimited particles that are naturally released from a cell and, unlike a cell, cannot replicate. Extracellular vesicles range in diameter from near the size of the smallest physically possible unilamellar liposome (around 20-30 nanometers) to as large as 10 microns or more, although the vast majority of extracellular vesicles are smaller than 200 nm. They may carry a cargo of proteins, nucleic acids, lipids, metabolites, and even organelles from the parent cell. Most cells that have been studies to date are thought to release extracellular vesicles, including some bacterial, fungal, and plant cells that are surrounded by cell walls. In some embodiments, the extracellular vesicles may comprise ectosomes, microvesicles (MV), microparticles (MP), exosomes, apoptotic bodies, large oncosomes, exophers, platelets, red blood cells, enveloped viruses, and exomeres. An “ectosome”, “microvesicle” (MV), or “microparticle” (MP) comprise extracellular vesicles released from the surface of cells, i.e., are of plasma membrane origin. Especially in the field of platelet research, microparticle has been standard nomenclature. Formation of ectosomes may in some cases result from directed processes, and in others from shear forces or adherence of the plasma membrane to a surface.

“Exosomes” are membrane bound extracellular vesicles that are produced in the endosomal compartment of most eukaryotic cells. Exosome biogenesis begins with pinching off of endosomal invaginations into the multivesicular body, forming intraluminal vesicles. If the multivesicular body fuses with the plasma membrane, the intraluminal vesicles are released as exosomes. In particular embodiments, the extracellular vesicles comprise exosomes.

“Apoptotic bodies” are extracellular vesicles that are released by dying cells undergoing apotosis. Since apoptotic cells tend to display phosphatidylserine (PS) in the outer bilayer of the cell membrane, apoptotic bodies tend to externalize PS, although other extracellular vesicles may do so. Apoptotic bodies may be quite large (microns in diameter) but may also measure in the submicron range.

In addition to the very large extracellular vesicles released during apoptosis, micron-sized extracellular vesicles may be produced by cancer cells, neurons, and other cells. When produced by cancer cells, these particles are termed “large oncosomes” and may reach 20 microns or more in diameter. These large extracellular vesicles are practically cells except without full nuclei. They contain a functional cytoskeleton and energy sources (mitochondria), and may be motile, contribution to metastasis. Another class of large extracellular vesicle has been observed in neurons of the model organism C. Elegans. When injected with a dye, neurons were observed to sequester the dye into a portion of the cell and release it in a large extracellular vesicle dubbed the “exopher”. This body was hypothesized to be a mechanism for disposal of unwanted cellular material. Technically, the platelets of certain vertebrates (which bud from megakaryocytes), as well as red blood cells (e.g., of adult humans) also fulfill the consensus definition of extracellular vesicles.

Enveloped viruses are a type of extracellular vesicle produced under the influence of viral infection. That is, the virion is composed of cellular membranes but contains proteins and nucleic acids produced from the viral genome. Some enveloped viruses can infect other cells even without a functional virion, when genomic material is transferred via extracellular vesicles. Certain non-enveloped viruses may also reproduce with assistant from extracellular vesicles.

In some embodiments, the medium comprises a biological fluid, for example blood (such as arterial or venous blood), cerebrospinal fluid, peritoneal fluid, pleural fluid, amniotic fluid, or lymphatic fluid. In typical embodiments, the biological fluid will comprise an extracellular fluid such as intravascular fluid, interstitial fluid, lymphatic fluid, or transcellular fluid. The fluid may be obtained from a biological organism, for example a human, by sampling methods that would be readily known in the art. In some embodiments, the biological fluid is blood (as would be obtained from arterial or venous blood sampling). In some embodiments, the biological fluid is cerebrospinal fluid (as would be obtained by a lumbar puncture). In some embodiments, the biological fluid is peritoneal fluid (as obtained by paracentesis) or pleural fluid (as obtained by thoracocentesis). In some embodiment, the biological fluid is amniotic fluid (as obtained by amniocentesis). The biological fluids as used in the present method may include, but are not limited to, cell culture medium, human and animal body fluids such as plasma, serum, urine, saliva, tears, perilymph fluid, milk and cerebrospinal fluid (CSF), and plant derived fluids.

The aqueous solution as used in the above methods may be any aqueous solution known to be acceptable used in the handling of biological material, for example such as cells. Among the acceptable aqueous solutions that may be employed are water, Ringer's solution, U.S.P. and isotonic sodium chloride solution. Further, the aqueous solution may comprise an acceptable buffer used in biology, for example a buffer selected from MES, Bis-Tris, ADA, ACES, PIPES, MOPSO, Bis-Tris Propane, BES, MOPS, TES, HEPES, DIPSO, MOBS, TAPSO, Tris, HEPPSO, POPSO, TEA, EPPS, Tricine, Gly-Gly, Bicine, HEPBS, TAPS, AMPD, TABS, AMPSO, CHES, CAPSO, APM, CAPS, and CABS. In some embodiments, the aqueous solution may comprise a phosphate buffer, a phosphate/citric acid buffer, a citric acid/sodium citrate buffer, an acetate/acetic acid buffer, an imidazole/hydrochloric acid buffer, or a carbonate/bicarbonate buffer.

The amount of the immunomagnetic composition required to be added to form the mixture can depend on a number of factors including, but not limited to, the volume of biological fluid being processed, the valency of peptide present conjugated to the magnetic particles, the expected abundance of the exosomes present, or combinations thereof. Too high of an amount of the immunomagnetic composition added to the mixture can induce non-specific binding. Too low of an amount of the immunomagnetic composition can result in low capture efficiency. One skilled in the art can determine the concentration of immunomagnetic particles.

The extracellular vesicles can be allowed to mix with the immunomagnetic composition for any period of time, e.g., seconds, minutes or hours. In some embodiments, the extracellular vesicles can be mixed with the immunomagnetic composition for at least about 1 minute, at least about 2 minutes, at least about 5 minutes, at least about 10 minutes, at least about 15 minutes, at least about 30 minutes, at least about 1 hour, at least about 2 hours or more. A person having ordinary skill in the art can readily determine an optimum mixing time, based on a number of factors, including, but not limited to, the affinity of the immunomagnetic composition with the extracellular vesicles, concentrations, mixing temperature and/or mixing speed. However, in one or more embodiments, the extracellular vesicles are mixed with the immunomagnetic composition for 1 hour or less.

Collection of the extracellular vesicles bound to the magnetic particles may be facilitated by applying a magnetic field to the magnetic particles. In some embodiments, the magnet has a strong enough magnetic field strength sufficient to create a magnetic field gradient to cause the extracellular vesicles bound to the magnetic particles to separate from the mixture. The immobilized extracellular vesicles can then be removed for further processing.

In some embodiments, the method may further comprise contacting the population of extracellular vesicles bound to the population of magnetic particles with an agent such that the agent is encapsulated within the extracellular vesicles. In some embodiments, the agent may be a therapeutic agent, for example a drug. In some embodiments, the agent comprises an anti-cancer agent. In other embodiments, the agent may comprise a vaccine, an immunotherapy agent, or a regenerative therapy agent.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

It is understood that disclosure herein of a therapeutic agent also disclosed pharmaceutically acceptable salt, pharmaceutically acceptable ester, pharmaceutically acceptable amide, prodrug forms, and derivates of the therapeutic agent.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

In some embodiments, the therapeutic agent may comprise an agent used to treat cancer, i.e., a cancer drug or anti-cancer agent. Exemplary cancer drugs can be selected from antimetabolite anti-cancer agents and antimitotic anti-cancer agents, and combinations thereof, to a subject. Various antimetabolite and antimitotic anti-cancer agents, including single such agents or combinations of such agents, may be employed in the methods and compositions described herein.

Antimetabolic anti-cancer agents typically structurally resemble natural metabolites, which are involved in normal metabolic processes of cancer cells such as the synthesis of nucleic acids and proteins. The antimetabolites, however, differ enough from the natural metabolites such that they interfere with the metabolic processes of cancer cells. In the cell, antimetabolites are mistaken for the metabolites they resemble, and are processed by the cell in a manner analogous to the normal compounds. The presence of the “decoy” metabolites prevents the cells from carrying out vital functions and the cells are unable to grow and survive. For example, antimetabolites may exert cytotoxic activity by substituting these fraudulent nucleotides into cellular DNA, thereby disrupting cellular division, or by inhibition of critical cellular enzymes, which prevents replication of DNA.

In one aspect, therefore, the antimetabolite anti-cancer agent is a nucleotide or a nucleotide analog. In certain aspects, for example, the antimetabolite agent may comprise purine (e.g., guanine or adenosine) or analogs thereof, or pyrimidine (cytidine or thymidine) or analogs thereof, with or without an attached sugar moiety.

Suitable antimetabolite anti-cancer agents for use in the present disclosure may be generally classified according to the metabolic process they affect, and can include, but are not limited to, analogues and derivatives of folic acid, pyrimidines, purines, and cytidine. Thus, in one aspect, the antimetabolite agent(s) is selected from the group consisting of cytidine analogs, folic acid analogs, purine analogs, pyrimidine analogs, and combinations thereof.

In one particular aspect, for example, the antimetabolite agent is a cytidine analog. According to this aspect, for example, the cytidine analog may be selected from the group consisting of cytarabine (cytosine arabinodside), azacitidine (5-azacytidine), and salts, analogs, and derivatives thereof.

In another particular aspect, for example, the antimetabolite agent is a folic acid analog. Folic acid analogs or antifolates generally function by inhibiting dihydrofolate reductase (DHFR), an enzyme involved in the formation of nucleotides; when this enzyme is blocked, nucleotides are not formed, disrupting DNA replication and cell division. According to certain aspects, for example, the folic acid analog may be selected from the group consisting of denopterin, methotrexate (amethopterin), pemetrexed, pteropterin, raltitrexed, trimetrexate, and salts, analogs, and derivatives thereof.

In another particular aspect, for example, the antimetabolite agent is a purine analog. Purine-based antimetabolite agents function by inhibiting DNA synthesis, for example, by interfering with the production of purine containing nucleotides, adenine and guanine which halts DNA synthesis and thereby cell division. Purine analogs can also be incorporated into the DNA molecule itself during DNA synthesis, which can interfere with cell division. According to certain aspects, for example, the purine analog may be selected from the group consisting of acyclovir, allopurinol, 2-aminoadenosine, arabinosyl adenine (ara-A), azacitidine, azathiprine, 8-aza-adenosine, 8-fluoro-adenosine, 8-methoxy-adenosine, 8-oxo-adenosine, cladribine, deoxycoformycin, fludarabine, gancylovir, 8-aza-guanosine, 8-fluoro-guanosine, 8-methoxy-guanosine, 8-oxo-guanosine, guanosine diphosphate, guanosine diphosphate-beta-L-2-aminofucose, guanosine diphosphate-D-arabinose, guanosine diphosphate-2-fluorofucose, guanosine diphosphate fucose, mercaptopurine (6-MP), pentostatin, thiamiprine, thioguanine (6-TG), and salts, analogs, and derivatives thereof.

In yet another particular aspect, for example, the antimetabolite agent is a pyrimidine analog. Similar to the purine analogs discussed above, pyrimidine-based antimetabolite agents block the synthesis of pyrimidine-containing nucleotides (cytosine and thymine in DNA; cytosine and uracil in RNA). By acting as “decoys,” the pyrimidine-based compounds can prevent the production of nucleotides, and/or can be incorporated into a growing DNA chain and lead to its termination. According to certain aspects, for example, the pyrimidine analog may be selected from the group consisting of ancitabine, azacitidine, 6-azauridine, bromouracil (e.g., 5-bromouracil), capecitabine, carmofur, chlorouracil (e.g. 5-chlorouracil), cytarabine (cytosine arabinoside), cytosine, dideoxyuridine, 3′-azido-3′-deoxythymidine, 3′-dideoxycytidin-2′-ene, 3 ‘-deoxy-3’-deoxythymidin-2′-ene, dihydrouracil, doxifluridine, enocitabine, floxuridine, 5-fluorocytosine, 2-fluorodeoxycytidine, 3-fluoro-3′-deoxythymidine, fluorouracil (e.g., 5-fluorouracil (also known as 5-FU), gemcitabine, 5-methylcytosine, 5-propynylcytosine, 5-propynylthymine, 5-propynyluracil, thymine, uracil, uridine, and salts, analogs, and derivatives thereof. In one aspect, the pyrimidine analog is other than 5-fluorouracil. In another aspect, the pyrimidine analog is gemcitabine or a salt thereof.

In certain aspects, the antimetabolite agent is selected from the group consisting of 5-fluorouracil, capecitabine, 6-mercaptopurine, methotrexate, gemcitabine, cytarabine, fludarabine, pemetrexed, and salts, analogs, derivatives, and combinations thereof. In other aspects, the antimetabolite agent is selected from the group consisting of capecitabine, 6-mercaptopurine, methotrexate, gemcitabine, cytarabine, fludarabine, pemetrexed, and salts, analogs, derivatives, and combinations thereof. In one particular aspect, the antimetabolite agent is other than 5-fluorouracil. In a particularly preferred aspect, the antimetabolite agent is gemcitabine or a salt or thereof (e.g., gemcitabine HCl (Gemzar®)).

Other antimetabolite anti-cancer agents may be selected from, but are not limited to, the group consisting of acanthifolic acid, aminothiadiazole, brequinar sodium, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, Wellcome EHNA, Merck & Co. EX-015, fazarabine, fludarabine phosphate, N-(2′-furanidyl)-5-fluorouracil, Daiichi Seiyaku FO-152, 5-FU-fibrinogen, isopropyl pyrrolizine, Lilly LY-188011; Lilly LY-264618, methobenzaprim, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, tiazofurin, Erbamont TIF, tyrosine kinase inhibitors, Taiho UFT and uricytin, among others.

In one aspect, the antimitotic agent is a microtubule inhibitor or a microtubule stabilizer. In general, microtubule stabilizers, such as taxanes and epothilones, bind to the interior surface of the beta-microtubule chain and enhance microtubule assembly by promoting the nucleation and elongation phases of the polymerization reaction and by reducing the critical tubulin subunit concentration required for microtubules to assemble. Unlike mictrotubule inhibitors, such as the vinca alkaloids, which prevent microtubule assembly, the microtubule stabilizers, such as taxanes, decrease the lag time and dramatically shift the dynamic equilibrium between tubulin dimers and microtubule polymers towards polymerization. In one aspect, therefore, the microtubule stabilizer is a taxane or an epothilone. In another aspect, the microtubule inhibitor is a vinca alkaloid.

In some embodiments, the therapeutic agent may comprise a taxane or derivative or analog thereof. The taxane may be a naturally derived compound or a related form, or may be a chemically synthesized compound or a derivative thereof, with antineoplastic properties. The taxanes are a family of terpenes, including, but not limited to paclitaxel (Taxol®) and docetaxel (Taxotere®), which are derived primarily from the Pacific yew tree, Taxus brevifolia, and which have activity against certain tumors, particularly breast and ovarian tumors. In one aspect, the taxane is docetaxel or paclitaxel. Paclitaxel is a preferred taxane and is considered an antimitotic agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions.

Also included are a variety of known taxane derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; deoxygenated paclitaxel compounds such as those described in U.S. Pat. No. 5,440,056; and taxol derivatives described in U.S. Pat. No. 5,415,869. As noted above, it further includes prodrugs of paclitaxel including, but not limited to, those described in WO 98/58927; WO 98/13059; and U.S. Pat. No. 5,824,701. The taxane may also be a taxane conjugate such as, for example, paclitaxel-PEG, paclitaxel-dextran, paclitaxel-xylose, docetaxel-PEG, docetaxel-dextran, docetaxel-xylose, and the like. Other derivatives are mentioned in “Synthesis and Anticancer Activity of Taxol Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elsevier, Amsterdam 1986), among other references. Each of these references is hereby incorporated by reference herein in its entirety.

Various taxanes may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267) (each of which is hereby incorporated by reference herein in its entirety), or obtained from a variety of commercial sources, including for example, Sigma-Aldrich Co., St. Louis, Mo.

Alternatively, the antimitotic agent can be a microtubule inhibitor; in one preferred aspect, the microtubule inhibitor is a vinca alkaloid. In general, the vinca alkaloids are mitotic spindle poisons. The vinca alkaloid agents act during mitosis when chromosomes are split and begin to migrate along the tubules of the mitosis spindle towards one of its poles, prior to cell separation. Under the action of these spindle poisons, the spindle becomes disorganized by the dispersion of chromosomes during mitosis, affecting cellular reproduction. According to certain aspects, for example, the vinca alkaloid is selected from the group consisting of vinblastine, vincristine, vindesine, vinorelbine, and salts, analogs, and derivatives thereof.

The antimitotic agent can also be an epothilone. In general, members of the epothilone class of compounds stabilize microtubule function according to mechanisms similar to those of the taxanes. Epothilones can also cause cell cycle arrest at the G2-M transition phase, leading to cytotoxicity and eventually apoptosis. Suitable epithiolones include epothilone A, epothilone B, epothilone C, epothilone D, epothilone E, and epothilone F, and salts, analogs, and derivatives thereof. One particular epothilone analog is an epothilone B analog, ixabepilone (Ixempra™)

In certain aspects, the antimitotic anti-cancer agent is selected from the group consisting of taxanes, epothilones, vinca alkaloids, and salts and combinations thereof. Thus, for example, in one aspect the antimitotic agent is a taxane. More preferably in this aspect the antimitotic agent is paclitaxel or docetaxel, still more preferably paclitaxel. In another aspect, the antimitotic agent is an epothilone (e.g., an epothilone B analog). In another aspect, the antimitotic agent is a vinca alkaloid.

Examples of cancer drugs that may be used in the present disclosure include, but are not limited to: thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as sunitimib and imatinib. Examples of additional cancer drugs include alkylating agents, antimetabolites, natural products, hormones and antagonists, and miscellaneous agents. Alternate names are indicated in parentheses. Examples of alkylating agents include nitrogen mustards such as mechlorethamine, cyclophosphainide, ifosfamide, melphalan sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, SFU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel, protein bound paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Alternate names and trade-names of these and additional examples of cancer drugs, and their methods of use including dosing and administration regimens, will be known to a person versed in the art.

In some aspects, the anti-cancer agent may comprise a chemotherapeutic agent. Suitable chemotherapeutic agents include, but are not limited to, alkylating agents, antibiotic agents, antimetabolic agents, hormonal agents, plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors, gene therapy agents, cell therapy, e.g., stem cells, or any combination thereof.

According to these aspects, the chemotherapeutic agent is selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride and combinations thereof. Each possibility represents a separate aspect of the invention.

According to certain aspects, the therapeutic agent may comprise a biologic drug, particularly an antibody. According to some aspects, the antibody is selected from the group consisting of cetuximab, anti-CD24 antibody, panitumumab and bevacizumab.

Therapeutic agents as used in the present disclosure may comprise peptides, proteins such as hormones, enzymes, antibodies, monoclonal antibodies, antibody fragments, monoclonal antibody fragments, and the like, nucleic acids such as aptamers, siRNA, DNA, RNA, antisense nucleic acids or the like, antisense nucleic acid analogs or the like, low-molecular weight compounds, or high-molecular-weight compounds, receptor agonists, receptor antagonists, partial receptor agonists, and partial receptor antagonists.

Additional representative therapeutic agents may include, but are not limited to, peptide drugs, protein drugs, desensitizing materials, antigens, factors, growth factors, anti-infective agents such as antibiotics, antimicrobial agents, antiviral, antibacterial, antiparasitic, antifungal substances and combination thereof, antiallergenics, steroids, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, anti-Alzheimer's agents, antihypertensive agents, beta-adrenergic blocking agents, alpha-adrenergic blocking agents, nutritional agents, and the benzophenanthridine alkaloids. The therapeutic agent can further be a substance capable of acting as a stimulant, a sedative, a hypnotic, an analgesic, an anticonvulsant, and the like.

Additional therapeutic agents may comprise CNS-active drugs, neuro-active drugs, inflammatory and anti-inflammatory drugs, renal and cardiovascular drugs, gastrointestinal drugs, anti-neoplastics, immunomodulators, immunosuppressants, hematopoietic agents, growth factors, anticoagulant, thrombolytic, antiplatelet agents, hormones, hormone-active agents, hormone antagonists, vitamins, ophthalmic agents, anabolic agents, antacids, anti-asthmatic agents, anti-cholesterolemic and anti-lipid agents, anti-convulsants, anti-diarrheals, anti-emetics, anti-manic agents, antimetabolite agents, anti-nauseants, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, laxatives, mineral supplements, mucolytic agents, neuromuscular drugs, peripheral vasodilators, psychotropics, stimulants, thyroid and anti-thyroid agents, tissue growth agents, uterine relaxants, vitamins, antigenic materials, and so on. Other classes of therapeutic agents include those cited in Goodman & Gilman's The Pharmacological Basis of Therapeutics (McGraw Hill) as well as therapeutic agents included in the Merck Index and The Physicians' Desk Reference (Thompson Healthcare).

Other therapeutic agents include androgen inhibitors, polysaccharides, growth factors (e.g., a vascular endothelial growth factor-VEGF), hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Further examples of therapeutic agents include, but are not limited to, peptide drugs, protein drugs, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, antiproliferatives, anti-VEGF agents, cardioactive agents, nonsteroidal anti-inflammatory agents, antiparkinsonian agents, antihypertensive agents, β-adrenergic blocking agents, nutritional agents, and the benzophenanthridine alkaloids. The agent can further be a substance capable of acting as a stimulant, sedative, hypnotic, analgesic, anticonvulsant, and the like.

Further representative therapeutic agents include but are not limited to analgesics such as acetaminophen, acetylsalicylic acid, and the like; anesthetics such as lidocaine, xylocaine, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, paclitaxel, taxol, and the like; anticoagulants such as heparin, warfarin, and the like; anticonvulsants such as phenyloin sodium, diazepam, and the like; antidepressants such as isocarboxazid, amoxapine, and the like; antihistamines such as diphenhydramine HCl, chlorpheniramine maleate, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B12, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxam®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The therapeutic agent can also be an immunomodulator, including, for example, cytokines, interleukins, interferon, colony stimulating factor, tumor necrosis factor, and the like; immunosuppressants such as rapamycin, tacrolimus, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcus neoformans, Histoplasma capsulatum, Candida albicans, Candida tropicalis, Nocardia asteroids, Rickettsia ricketsii, Rickettsia typhi, Mycoplasma pneumoniae, Chlamydia psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In a further specific aspect, the therapeutic agent can comprise an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Timidazole, or a combination thereof. In one aspect, the therapeutic agent can be a combination of Rifampicin (Rifampin in U.S.) and Minocycline.

Growth factors useful as therapeutic agents include, but are not limited to, transforming growth factor-α (“TGF-α”), transforming growth factors (“TGF-β”), platelet-derived growth factors (“PDGF”), fibroblast growth factors (“FGF”), including FGF acidic isoforms 1 and 2, FGF basic form 2 and FGF 4, 8, 9 and 10, nerve growth factors (“NGF”) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, beta1, beta2, beta3, skeletal growth factor, bone matrix derived growth factors, and bone derived growth factors and mixtures thereof.

Cytokines useful as therapeutic agents include, but are not limited to, cardiotrophin, stromal cell derived factor, macrophage derived chemokine (MDC), melanoma growth stimulatory activity (MGSA), macrophage inflammatory proteins 1 alpha (MIP-1alpha), 2, 3 alpha, 3 beta, 4 and 5, IL-1, IL-2, IL-3, IL-4, IL-5, IL-6, IL-7, IL-8, IL-9, IL-10, IL-11, IL-12, IL-13, TNF-α, and TNF-β. Immunoglobulins useful in the present disclosure include, but are not limited to, IgG, IgA, IgM, IgD, IgE, and mixtures thereof. Some preferred growth factors include VEGF (vascular endothelial growth factor), NGFs (nerve growth factors), PDGF-AA, PDGF-BB, PDGF-AB, FGFb, FGFa, and BGF.

Other molecules useful as therapeutic agents include but are not limited to growth hormones, leptin, leukemia inhibitory factor (LIF), tumor necrosis factor alpha and beta, endostatin, thrombospondin, osteogenic protein-1, bone morphogenetic proteins 2 and 7, osteonectin, somatomedin-like peptide, osteocalcin, interferon alpha, interferon alpha A, interferon beta, interferon gamma, interferon 1 alpha, and interleukins 2, 3, 4, 5 6, 7, 8, 9, 10, 11, 12,13, 15, 16, 17 and 18.

In some embodiments, the population of extracellular vesicles may be loaded as described in the methods herein with a vaccine. The vaccine may comprise an antigen which is selected from a human pathogen, such as a viral or bacterial antigen; or the antigen may be a human self-antigen for the treatment of a chronic disorder such as allergy, cancer, autoimmune disease, Alzheimer's disease and others.

In some embodiments, the vaccine may comprise an antigen or antigenic composition capable of eliciting an immune response against a human pathogen, which antigen or antigenic composition is derived from HIV-1, (such as tat, nef, gp120 or gp160), human herpes viruses (ISV), such as gD or derivatives thereof or Immediate Early protein such as ICP27 from HSV1 or HSV2, cytomegalovirus (CMV (esp Human)(such as gB or derivatives thereof), Rotavirus (including live-attenuated viruses), Epstein Barr virus (such as gp350 or derivatives thereof), Varicella Zoster Virus (VZV, such as gpI, II and 1E63), or from a hepatitis virus such as hepatitis B virus (for example Hepatitis B Surface antigen or a derivative thereof), hepatitis A virus (HAV), hepatitis C virus and hepatitis E virus, or from other viral pathogens, such as paramyxoviruses: Respiratory Syncytial virus (RSV, such as F and G proteins or derivatives thereof), parainfluenza virus, measles virus, mumps virus, human papilloma viruses (HPV, for example BPV6, 11, 16, 18), flaviviruses (e.g. Yellow Fever Virus, Dengue Virus, Tick-borne encephalitis virus, Japanese Encephalitis Virus) or Influenza virus (whole live or inactivated virus, split influenza virus, grown in eggs or MDCK cells, or whole flu virosomes (as described by R. Gluck, Vaccine, 1992, 10, 915-920) or purified or recombinant proteins thereof, such as HA, NP, NA, or M proteins, or combinations thereof), or derived from bacterial pathogens such as Neisseria spp, including N. gonorrhea and N. meningitidis (for example capsular polysaccharides and conjugates thereof, transferrin-binding proteins, lactoferrin binding proteins, PilC, adhesins); S. pyogenes (for example M proteins or fragments thereof, C5A protease, lipoteichoic acids), S. agalactiae, S. mutans; H ducreyi; Moraxella spp, including M. catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, —B or —C), M. bovis, M. leprae, M. avium, M. paratuberculosis, M. smegmatis; Legionella spp, including L. pneumophila, Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridiuni spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. micron; Trypanosoma spp., including T. cruzi; Giardia spp., including G. lamblia; Leshmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

Other preferred specific antigens for M. tuberculosis are for example Th Ra12, Tb H9, Th Ra35, Tb38-1, Erd 14, DPV, MTI, MSL, mTTC2 and hTCC1 (WO 99/51748). Proteins for M. tuberculosis also include fusion proteins and variants thereof where at least two, preferably three polypeptides of M. tuberculosis are fused into a larger protein. Preferred fusions include Ra12-TbH9-Ra35, Erd14-DPV-MTI, DPV-MTI-MSL, Erd14-DPV-MTI-MSL-mTCC2, Erd14-DPV-MTI-MSL, DPV-MTI-MSL-mTCC2, TbH9-DPV-MTI (WO 99/51748).

Most preferred antigens for Chlamydia include for example the High Molecular Weight Protein (HWMP) (WO 99/17741), ORF3 (EP 366 412), and putative membrane proteins (Pmps). Other Chlamydia antigens of the vaccine formulation can be selected from the group described in WO 99/28475.

Preferred bacterial vaccines comprise antigens derived from Streptococcus spp, including S. pneumoniae (for example capsular polysaccharides and conjugates thereof, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Particularly preferred pneumococcal vaccines are those described in WO 00/56539. Other preferred bacterial vaccines comprise antigens derived from Haemophilus spp., including H. influenzae type B (“Hib”, for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof.

Derivatives of Hepatitis B Surface antigen are well known in the art and include, inter alia, those PreS1, PreS2 S antigens set forth described in European Patent applications EP-A-414 374; EP-A-0304 578, and EP 198-474. In one preferred aspect the vaccine formulation of the invention comprises the HIV-1 antigen, gp120, especially when expressed in CHO cells. In a further embodiment, the vaccine formulation of the invention comprises gD2t as hereinabove defined.

In a preferred embodiment of the present invention vaccines containing the claimed adjuvant comprise antigen derived from the Human Papilloma Virus (IPV) considered to be responsible for genital warts (HPV 6 or HPV 11 and others), and the HPV viruses responsible for cervical cancer (HPV16, HPV18 and others).

Particularly preferred forms of genital wart prophylactic, or therapeutic, vaccine comprise L1 particles or capsomers, and fusion proteins comprising one or more antigens selected from the HPV 6 and HPV 1 proteins E6, E7, L1, and L2.

The most preferred forms of fusion protein are: L2E7 as disclosed in WO 96/26277, and proteinD(1/3)-E7 disclosed in GB 9717953.5 (PCT/EP98/05285).

A preferred HPV cervical infection or cancer, prophylaxis or therapeutic vaccine, composition may comprise HPV 16 or 18 antigens. For example, L1 or L2 antigen monomers, or L1 or L2 antigens presented together as a virus like particle (VLP) or the L1 alone protein presented alone in a VLP or caposmer structure. Such antigens, virus like particles and capsomer are per se known. See for example WO94/00152, WO94/20137, WO94/05792, and WO93/02184.

Additional early proteins may be included alone or as fusion proteins such as E7, E2 or preferably E5 for example; particularly preferred embodiments of this includes a VLP comprising L1E7 fusion proteins (WO 96/11272).

Particularly preferred HPV 16 antigens comprise the early proteins E6 or E7 in fusion with a protein D carrier to form Protein D-E6 or B7 fusions from HPV 16, or combinations thereof; or combinations of E6 or E7 with L2 (WO 96/26277).

Alternatively the HPV 16 or 18 early proteins E6 and E7, may be presented in a single molecule, preferably a Protein D-E6/E7 fusion. Such vaccine may optionally contain either or both E6 and E7 proteins from HPV 18, preferably in the form of a Protein D-E6 or Protein D-E7 fusion protein or Protein D E6/E7 fusion protein.

The vaccine as enclosed in the extracellular vesicles described herein may additionally comprise antigens from other HPV strains, preferably from strains HPV 31 or 33.

The vaccines as used in the extracellular vesicles described herein may further comprise antigens derived from parasites that cause Malaria. For example, preferred antigens from Plasmodia falciparum include RTS,S and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. Its full structure is disclosed in the International Patent Application No. PCT/EP92/02591, published under Number WO 93/10152 claiming priority from UK patent application No. 9124390.7. When expressed in yeast RTS is produced as a lipoprotein particle, and when it is co-expressed with the S antigen from HBV it produces a mixed particle known as RTS,S. TRAP antigens are described in the International Patent Application No. PCT/GB89/00895, published under WO 90/01496. A preferred embodiment of the present invention is a Malaria vaccine wherein the antigenic preparation comprises a combination of the RTS,S and TRAP antigens. Other plasmodia antigens that are likely candidates to be components of a multistage Malaria vaccine are P. faciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.

In some embodiments, the agent may comprise a peptide, an siRNA, a microRNA, one or more components of a Crispr-Cas9 system, a plasmid, or an enzyme.

In some embodiments, the method may further comprise contacting the population of extracellular vesicles bound to the population of magnetic particles with a surface modifying agent. In some embodiments, each extracellular vesicle comprises a lipid bilayer, and the surface modifying agent inserts within the lipid bilayer. In some embodiments, the surface modifying agent chemically modifies a component of the lipid bilayer, for example a membrane lipid or protein.

In some embodiments, the method may further comprise releasing the population of extracellular vesicles from the magnetic particles by adjusting the pH within a pH range of about 7 to about 9.

In some aspects, a population of extracellular vesicles is also provided that has been isolated using the methods described herein. Further provided is a pharmaceutical composition comprising a population of extracellular vesicles isolated using the methods described herein and a pharmaceutically acceptable excipient. “Excipients” include any and all solvents, diluents or other liquid vehicles, dispersion or suspension aids, surface active agents, isotonic agents, thickening or emulsifying agents, preservatives, solid binders, lubricants, and the like, as suited to the particular application desired. General considerations in formulation and/or manufacture can be found, for example, in Remington's Pharmaceutical Sciences, Sixteenth edition, E. W. Martin (Mack Publishing Co., Easton, Pa. 1980) and Remington: The Science and Practice of Pharmacy, 21^{st} Edition (Lippincott Williams and Wilkins, 2005).

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

### Example 1. On Demand Capture and Release of EVs and Exosomes Using Immunogenic Beads Conjugated with Insertion Peptides

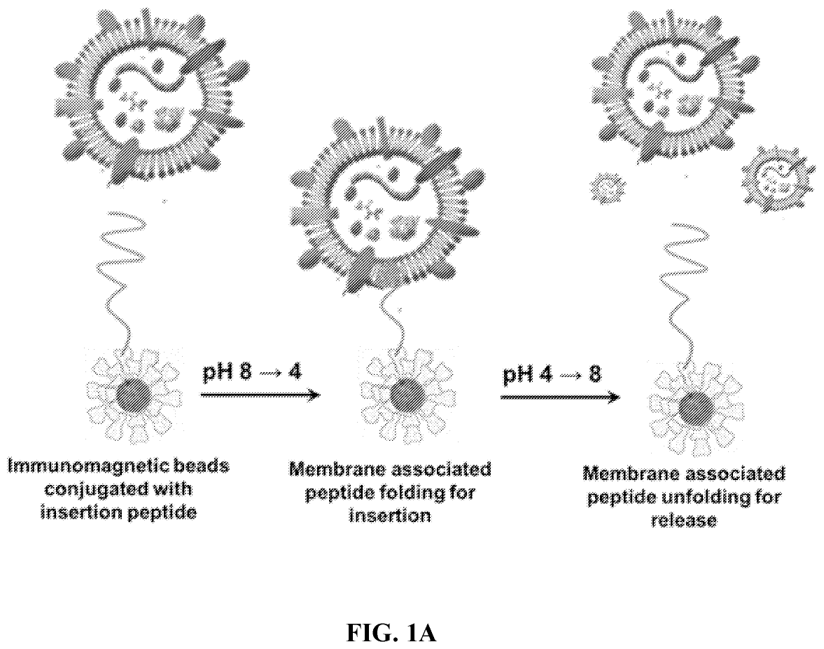

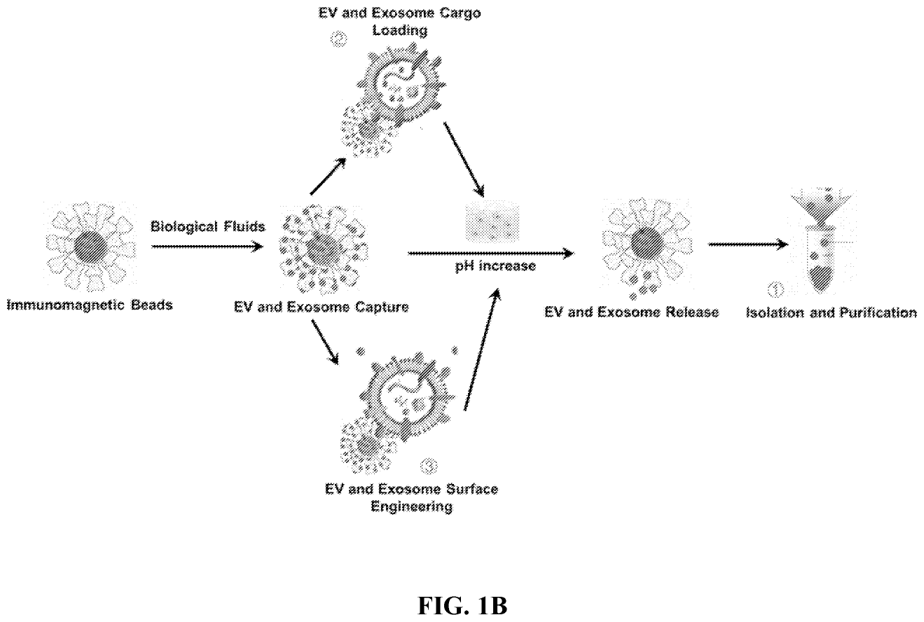

An immunomagnetic bead approach conjugated with insertion peptide was developed for pH-controlled, on demand capture and release of EVs and exosomes from biological fluids, as illustrated in FIG. 1. The biological fluids include but not limited to cell culture medium, human and animal body fluids such as plasma, serum, urine, saliva, tear, perilymph fluid, milk and cerebrospinal fluid (CSF) etc., and plant derived fluids.

Immunomagnetic beads are nanographene-PDA modified surface, which is used to conjugate with insertion peptide via amine (peptide)—NHS (beads surface) conjugation chemistry or biotin peptide conjugated with streptavidin immunomagnetic beads. Therefore, the insertion peptides immobilized on immunomagnetic beads surface will contact EVs and exosome lipid bilayer membrane surfaces via changing the solution pH from 8 to 4 or reverse for triggering the capture and release of EVs and Exosomes. Such a capture and release process is controllable and on demand. The insertion peptide can form a transmembrane helical orientation at low pH of around 4 and the C-terminus of peptide inserts across the lipid bilayer membrane spontaneously. By increasing pH to 8 rapidly, this process can be reversed in a few seconds and the inserted peptide will unfold and release out from lipid bilayer membrane spontaneously, as illustrated in FIG. 1A. Although this insertion peptide has been used to target and tether cargo molecules to the surfaces of cells in low pH environments of acidic diseased tissues, there is not any work reported for conjugation with immunomagnetic beads utilized for capture and release of EVs and exosomes. The insertion peptide sequences AEQNPIYWARYADWLFTTPLLLLDLALLVDADEGT (SEQ. ID NO: 1) and its Pro to Ala variant AEQNPIYWARYADWLFTTALLLLDLALLVDADEGT (SEQ. ID NO: 2) are prepared by solid-phase peptide synthesis using Fmoc (9-fluorenylmethyloxycarbonyl) chemistry and purified by reverse phase chromatography.

As illustrated in FIG. 1B, after capture of EVs and exosomes containing lipid bilayer membranes via immunomagnetic beads conjugated insertion peptide, the surface engineering and loading approaches can be introduced, such as encapsulation of drugs, genes and bioactive therapeutics. Because of the immobilization of captured EVs and exosomes via immunomagnetic beads and magnetic trapping, the streamlined manipulation can be introduced without losing EVs and exosomes. Such sequential steps including capture, loading, and release can be integrated as a streamlined operation in microfluidic device or sample tubes, and harvest therapeutic EVs and exosomes at the outlet for serving as vaccine platform, drug delivery platform, immunotherapy agents, as well as regenerative treatment agents. Utilizing insertion peptide as the capture agent is superior for capturing total EVs and exosomes, as long as the lipid bilayer is presented. So there is no bias as antibody capture approach which only selects affinity antigens from a portion of EVs and exosomes. Additionally, the release of captured EVs and exosomes is achieved by unfolding of inserted peptide, therefore, there is no any affinity tag or capture tag left over on the surfaces of EVs and exosomes. The obtained EVs and exosomes are original, pure and intact.

The immunomagnetic insertion peptide beads are superior for isolating and purifying original and intact EVs and exosomes. It is also easy to manipulate and integrate, and highly compatible with other sample preparation methods handling EVs and exosomes.

### Example 2. Isolation and Engineering of Highly Homogeneous, Inhalable Mesenchymal Stem Cell Secreted Exosomes (MSC-Exos) with Specific Lung Tissue Targeting

Compared to live MSC cell therapy, MSCs-Exos have longer storage stability which allows safe transportation and delayed therapeutic use[1]. Most importantly, the administration of MSCs may result in aggregating or clumping in the injured microcirculation and carries the risk of mutagenicity and oncogenicity, which does not exist by treating with MSCs-Exos as a cell-free therapy approach[2]. Substantial preclinical lung disease models, including ARDS, asthma, emphysema, and pulmonary arterial hypertension (PAH), have demonstrated that systemic administration of MSC-Exos showed anti-inflammatory, antifibrotic, and microbicidal effects[3-7]. However, preparing well-defined homogeneous MSC-Exos delivered to lung specifically is challenging. It has been observed that not all extracellular vesicles from MSC cells are equally regenerative as exosomes [8, 9], neither targeting lung specifically[10, 11]. In order to overcome these challenges and speed up the clinical translation, we propose to develop a fast deployable, lung-targeting MSC-Exos inhalation therapy for use in treating pulmonary inflammation and fibrosis.

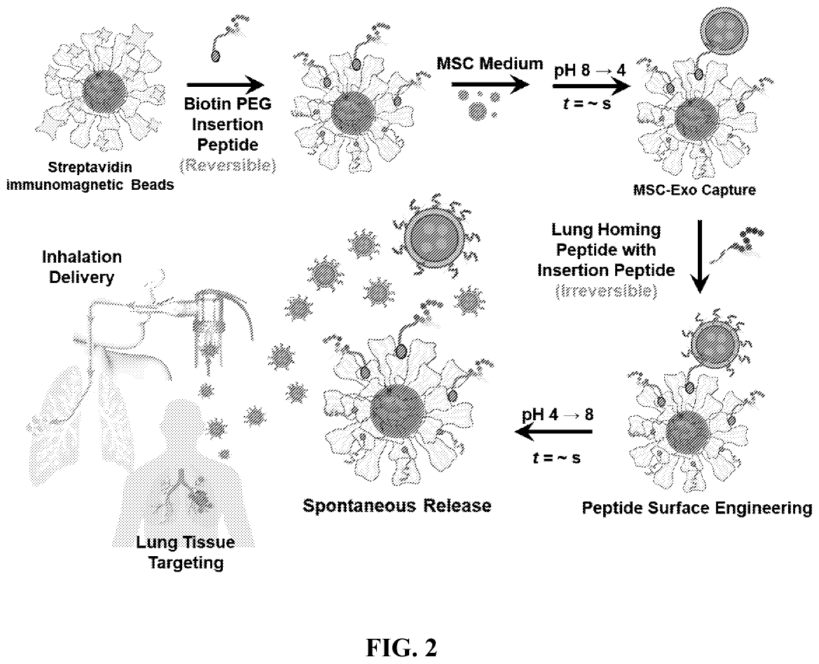

As shown in FIG. 2, a biotinylated pH-responsive insertion peptide can be conjugated with streptavidin-functionalized immunomagnetic beads, which can isolate, engineer and release intact, highly homogeneous therapeutic exosomes in a streamlined workflow for scale up, without introducing exogenous contamination. The biotin PEG insertion peptide (PH responsive) can bind to streptavidin on the immunomagnetic bead surface. The reversible pH-responsive peptide (sequence: ACDDQNPWRAYLDLLFPTDTLLLDLLW; SEQ. ID NO: 21) can form transmembrane helical orientation at pH 4 and the C-terminus of peptide inserts across the lipid bilayer membrane [12-15] of exosomes spontaneously in a speed of a few seconds. Meanwhile, the irreversible pH-responsive insertion peptide[12, 16, 17] (sequence: YQCKVYTGVYPFMWGGAYCFC; SEQ. ID NO: 22) conjugated with lung homing peptide (GFE motif) [18, 19] can be introduced to decorate the captured exosomes with the ability of lung epithelial tissue targeting. By increasing pH to 8, the reversible pH-responsive insertion peptide will unfold and release out from the lipid bilayer membrane of exosomes spontaneously, while remain the decoration of lung homing peptides introduced by irreversible insertion peptide. Such insertion and release process are controllable and on demand, and spontaneously occurred within few seconds[20]. Therefore, this approach offers unmatched advantages to: 1) release intact, specific exosomes populations without introducing exogenous capture probes; 2) enable the integration of surface engineering and drug loading for producing therapeutic functional exosomes; 3) enable the development of transferrable, standardized production protocols via precise control of electromagnetic field and buffer pH; 4) offer simple, rapid, and streamlined workflow which is compatible with manufacturing bioreactor for scale up. Additionally, surface engineering of lung tissue homing peptides on captured exosomes can be integrated in one streamlined workflow by precise control of pH and electro-magnetic field, which enables the preparation of homogeneous therapeutic exosomes at scale for clinical translation and improving lung delivery specificity.

It has been observed that MSC-Exos could attenuate and resolve pulmonary fibrosis by reestablishing normal alveolar structure and decreasing both collagen accumulation and myofibroblast proliferation[21, 22], which showed superior therapeutic benefits for preventing and reducing pulmonary inflammation and fibrosis in animal models[23, 24]. However, MSC cells secret a mixture of extracellular vesicles which are not equally regenerative as exosomes[8, 9], neither targeting lung specifically[10, 11], which is the biggest roadblock for delivering effective exosome-based therapy. For maximizing clinical therapeutic outcomes, inhalation offers direct delivery to lung with minimal systemic exposure and side effects[25, 26]. Thus, exosome inhalation therapy could substantially improve the delivery specificity and efficacy.

### REFERENCES

[1] J. Phan, P. Kumar, D. Hao, K. Gao, D. Farmer, A. Wang, Engineering mesenchymal stem cells to improve their exosome efficacy and yield for cell-free therapy, J Extracell Vesicles 7(1) (2018) 1522236.
[2] P. Wu, B. Zhang, H. Shi, H. Qian, W. Xu, MSC-exosome: A novel cell-free therapy for cutaneous regeneration, Cytotherapy 20(3) (2018) 291-301.
[3] G. Hansmann, A. Fernandez-Gonzalez, M. Aslam, S. H. Vitali, T. Martin, S. A. Mitsialis, S. Kourembanas, Mesenchymal stem cell-mediated reversal of bronchopulmonary dysplasia and associated pulmonary hypertension, Pulm Circ 2(2) (2012) 170-81.
[4] L. Ionescu, R. N. Byrne, T. van Haaften, A. Vadivel, R. S. Alphonse, G. J. Rey-Parra, G. Weissmann, A. Hall, F. Eaton, B. Thebaud, Stem cell conditioned medium improves acute lung injury in mice: in vivo evidence for stem cell paracrine action, Am J Physiol Lung Cell Mol Physiol 303(11) (2012) L967-77.
[5] L. I. Ionescu, R. S. Alphonse, N. Arizmendi, B. Morgan, M. Abel, F. Eaton, M. Duszyk, H. Vliagoftis, T. R. Aprahamian, K. Walsh, B. Thebaud, Airway delivery of soluble factors from plastic-adherent bone marrow cells prevents murine asthma, Am J Respir Cell Mol Biol 46(2) (2012) 207-16.
[6] R. P. Sutsko, K. C. Young, A. Ribeiro, E. Tones, M. Rodriguez, D. Hehre, C. Devia, I. McNiece, C. Suguihara, Long-term reparative effects of mesenchymal stem cell therapy following neonatal hyperoxia-induced lung injury, Pediatr Res 73(1) (2013) 46-53.
[7] F. F. Cruz, Z. D. Borg, M. Goodwin, D. Sokocevic, D. E. Wagner, A. Coffey, M. Antunes, K. L. Robinson, S. A. Mitsialis, S. Kourembanas, K. Thane, A. M. Hoffman, D. H. McKenna, P. R. Rocco, D. J. Weiss, Systemic Administration of Human Bone Marrow-Derived Mesenchymal Stromal Cell Extracellular Vesicles Ameliorates Aspergillus Hyphal Extract-Induced Allergic Airway Inflammation in Immunocompetent Mice, Stem Cells Transl Med 4(11) (2015) 1302-16.
[8] H. S. Joo, J. H. Suh, H. J. Lee, E. S. Bang, J. M. Lee, Current Knowledge and Future Perspectives on Mesenchymal Stem Cell-Derived Exosomes as a New Therapeutic Agent, Int J Mol Sci 21(3) (2020).
[9] K. Yin, S. Wang, R. C. Zhao, Exosomes from mesenchymal stem/stromal cells: a new therapeutic paradigm, Biomark Res 7 (2019) 8.
[10] G. Zhao, Y. Ge, C. Zhang, L. Zhang, J. Xu, L. Qi, W. Li, Progress of Mesenchymal Stem Cell-Derived Exosomes in Tissue Repair, Curr Pharm Des (2020).
[11] T. Zhao, F. Sun, J. Liu, T. Ding, J. She, F. Mao, W. Xu, H. Qian, Y. Yan, Emerging Role of Mesenchymal Stem Cell-derived Exosomes in Regenerative Medicine, Curr Stem Cell Res Ther 14(6) (2019) 482-494.
[12] R. Saar-Dover, A. Ashkenazi, Y. Shai, Peptide interaction with and insertion into membranes, Methods Mol Biol 1033 (2013) 173-83.
[13] D. Weerakkody, O. A. Andreev, Y. K. Reshetnyak, Insertion into lipid bilayer of truncated pHLIP((R))peptide, Biochem Biophys Rep 8 (2016) 290-295.
[14] T. T. Tapmeier, A. Moshnikova, J. Beech, D. Allen, P. Kinchesh, S. Smart, A. Harris, A. McIntyre, D. M. Engelman, O. A. Andreev, Y. K. Reshetnyak, R. J. Muschel, The pH low insertion peptide pHLIP Variant 3 as a novel marker of acidic malignant lesions, Proc Natl Acad Sci USA 112(31) (2015) 9710-5.
[15] V. P. Nguyen, D. S. Alves, H. L. Scott, F. L. Davis, F. N. Barrera, A Novel Soluble Peptide with pH-Responsive Membrane Insertion, Biochemistry 54(43) (2015) 6567-75.
[16] F. T. Arce, H. Jang, S. Ramachandran, P. B. Landon, R. Nussinov, R. Lal, Polymorphism of amyloid beta peptide in different environments: implications for membrane insertion and pore formation, Soft Matter 7(11) (2011) 5267-5273.
[17] J. Tang, F. Gai, Dissecting the membrane binding and insertion kinetics of a pHLIP peptide, Biochemistry 47(32) (2008) 8250-2.
[18] A. Koivistoinen, Ilonen, II, K. Punakivi, J. V. Rasanen, H. Helin, E. I. Sihvo, M. Bergman, J. A. Salo, A novel peptide (Thx) homing to non-small cell lung cancer identified by ex vivo phage display, Clin Transl Oncol 15(6) (2013) 492-8.
[19] G. E. Holt, P. Daftarian, Non-small-cell lung cancer homing peptide-labeled dendrimers selectively transfect lung cancer cells, Immunotherapy 10(16) (2018) 1349-1360.
[20] G. Slaybaugh, D. Weerakkody, D. M. Engelman, O. A. Andreev, Y. K. Reshetnyak, Kinetics of pHLIP peptide insertion into and exit from a membrane, Proc Natl Acad Sci USA (2020).
[21] N. Mansouri, G. R. Willis, A. Fernandez-Gonzalez, M. Reis, S. Nassiri, S. A. Mitsialis, S. Kourembanas, Mesenchymal stromal cell exosomes prevent and revert experimental pulmonary fibrosis through modulation of monocyte phenotypes, JCI Insight 4(21) (2019).
[22] M. S. Njock, J. Guiot, M. A. Henket, O. Nivelles, M. Thiry, F. Dequiedt, J. L. Corhay, R. E. Louis, I. Struman, Sputum exosomes: promising biomarkers for idiopathic pulmonary fibrosis, Thorax 74(3) (2019) 309-312.
[23] P. C. Dinh, D. Paudel, H. Brochu, K. D. Popowski, M. C. Gracieux, J. Cores, K. Huang, M. T. Hensley, E. Harrell, A. C. Vandergriff, A. K. George, R. T. Barrio, S. Hu, T. A. Allen, K. Blackburn, T. G. Caranasos, X. Peng, L. V. Schnabel, K. B. Adler, L. J. Lobo, M. B. Goshe, K. Cheng, Inhalation of lung spheroid cell secretome and exosomes promotes lung repair in pulmonary fibrosis, Nat Commun 11(1) (2020) 1064.
[24] M. Y. Yao, W. H. Zhang, W. T. Ma, Q. H. Liu, L. H. Xing, G. F. Zhao, microRNA-328 in exosomes derived from M2 macrophages exerts a promotive effect on the progression of pulmonary fibrosis via FAM13A in a rat model, Exp Mol Med 51(6) (2019) 1-16.
[25] L. Willis, D. Hayes, Jr., H. M. Mansour, Therapeutic liposomal dry powder inhalation aerosols for targeted lung delivery, Lung 190(3) (2012) 251-62.
[26] A. Nieto-Orellana, H. Li, R. Rosiere, N. Wauthoz, H. Williams, C. J. Monteiro, C. Bosquillon, N. Childerhouse, G. Keegan, D. Coghlan, G. Mantovani, S. Stolnik, Targeted PEG-poly(glutamic acid) complexes for inhalation protein delivery to the lung, J Control Release 316 (2019) 250-262.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. An immunomagnetic composition comprising a population of magnetic particles, wherein each magnetic particle is conjugated to at least one pH responsive extracellular vesicle-binding peptide.

2.-4. (canceled)

5. The immunomagnetic composition of claim 1, wherein the population of magnetic particles has an average particle size ranging from about 1 nm to about 100 microns.

6. (canceled)

7. The immunomagnetic composition of claim 1, wherein the magnetic particle comprises a magnetic element selected from iron, nickel, and cobalt, or oxide compounds thereof.

8. The immunomagnetic composition of claim 1, wherein a graphene oxide nanomaterial is covalently bound to a surface of the magnetic particle.

9. (canceled)

10. The immunomagnetic composition of claim 1, wherein the peptide is conjugated or covalently bound to a surface of the magnetic particle.

11.-13. (canceled)

14. The immunomagnetic composition of claim 1, wherein the magnetic particle comprises one or more streptavidin groups.

15. The immunomagnetic composition of claim 14, wherein the peptide comprises a biotinylated residue, and wherein the biotinylated residue is bound to one streptavidin group.

16. The immunomagnetic composition of claim 1, wherein the peptide comprises an amino acid sequence having at least 65%80% identity with an amino acid sequence selected from SEQ ID NO: 1 to 21.

17.-19. (canceled)

20. The immunomagnetic composition of claim 1, wherein the peptide comprises an amino acid sequence selected from SEQ ID NO: 1 to 21.

21. The immunomagnetic composition of claim 1, wherein the peptide consists of an amino acid sequence having at least 80% identity with an amino acid sequence selected from SEQ ID NO: 1 to 21.

22.-24. (canceled)

25. The immunomagnetic composition of claim 1, wherein the peptide consists of an amino acid sequence selected from SEQ ID NO: 1 to 21.

26. A method for isolating a population of extracellular vesicles from a medium the method comprising:
contacting the medium with an immunomagnetic composition of claim 1 and optionally an aqueous solution to form a mixture;
adjusting the pH of the mixture to within a range of about 3 to about 5 to bind the population of magnetic particles to the population of extracellular vesicles; and
collecting the population of extracellular vesicles bound to the magnetic particles by applying a magnetic field.

27. The method of claim 26, wherein the extracellular vesicles comprise ectosomes, microvesicles (MV), microparticles (MP), exosomes, apoptotic bodies, large oncosomes, exophers, enveloped viruses, and exomeres.

28.-29. (canceled)

30. The method of claim 26, wherein the biological fluid comprises cell culture medium, plasma, serum, urine, saliva, tears, perilymph fluid, milk, cerebrospinal fluid, blood, or a plant derived fluid.

31. The method of claim 26, further comprising contacting the population of extracellular vesicles bound to the population of magnetic particles with an agent such that the agent is encapsulated within the extracellular vesicles.

32. The method of claim 31, wherein the agent comprises a therapeutic agent, an anti-cancer agent, a vaccine, an immunotherapy agent, or a regenerative therapy agent.

33.-36. (canceled)

37. The method of claim 26, further comprising contacting the population of extracellular vesicles bound to the population of magnetic particles with a surface modifying agent.

38.-40. (canceled)

41. The method of claim 39, wherein the component of the lipid bilayer comprises a membrane protein.

42. The method of claim 26, further comprising releasing the population of extracellular vesicles from the magnetic particles by adjusting the pH within a pH range of about 7 to about 9.

43. A population of extracellular vesicles isolated by the method of claim 26.

44. A pharmaceutical composition comprising the population of extracellular vesicles of claim 43 and a pharmaceutically acceptable excipient.

45. A kit comprising:
an immunomagnetic composition of claim 1; and
an aqueous solution for dispersing the immunomagnetic composition.


SEQUENCE LISTING (22 sequences)

SEQ ID NO: 1 - protein, 35 aa, Artificial Sequence
  Synthetic construct
AEQNPIYWARYADWLFTTPLLLLDLALLVDADEGT

SEQ ID NO: 2 - protein, 35 aa, Artificial Sequence
  Synthetic construct
AEQNPIYWARYADWLFTTALLLLDLALLVDADEGT

SEQ ID NO: 3 - protein, 37 aa, Artificial Sequence
  Synthetic construct
AEQNPIYWARYAGLAWLFTTPLLLLDLALLVDADEGT

SEQ ID NO: 4 - protein, 38 aa, Artificial Sequence
  Synthetic construct
AEDNPIYWARYAGLAWLFTTPLLLAADLALLVDADEGT

SEQ ID NO: 5 - protein, 26 aa, Artificial Sequence
  Synthetic construct
ADDQDPWRAYLDLLFPTDTLLLDLLW

SEQ ID NO: 6 - protein, 28 aa, Artificial Sequence
  Synthetic construct
ADDQNPWRAYLGLALLFPTDTLLLDLLW

SEQ ID NO: 7 - protein, 30 aa, Artificial Sequence
  Synthetic construct
GEEQNPWLGAYLDLLFPLELLGLLELGLWG

SEQ ID NO: 8 - protein, 34 aa, Artificial Sequence
  Synthetic construct
GLAGLAGLLGLEGLLGLPLGLLELLWLGLELEGN

SEQ ID NO: 9 - protein, 36 aa, Artificial Sequence
  Synthetic construct
GGEQNPIYWARYADWLFTTPLLLLDLALLVDADEGT

SEQ ID NO: 10 - protein, 36 aa, Artificial Sequence
  Synthetic construct
ACEQNPIYWARYADWLFTTPLLLLDLALLVDADEGT

SEQ ID NO: 11 - protein, 38 aa, Artificial Sequence
  Synthetic construct
ACAKEQNPIYWARYADWLFTTPLLLLDLALLVDADECT

SEQ ID NO: 12 - protein, 40 aa, Artificial Sequence
  Synthetic construct
ACAKEQNPIYWARYAGLAWLFTTPLLLLDLALLVDADECT

SEQ ID NO: 13 - protein, 42 aa, Artificial Sequence
  Synthetic construct
ACAKEQNPIYWARYAGLAWLFTTPLLLLAADLALLVDADECT

SEQ ID NO: 14 - protein, 28 aa, Artificial Sequence
  Synthetic construct
ACDDQNPWRAYLDLLFPTDTLLLDLLWA

SEQ ID NO: 15 - protein, 28 aa, Artificial Sequence
  Synthetic construct
ADDQNPWRAYLDLLFPTDTLLLDLLWCA

SEQ ID NO: 16 - protein, 29 aa, Artificial Sequence
  Synthetic construct
ACDDQNPWRAYLGLALFPTDTLLLDLLWG

SEQ ID NO: 17 - protein, 30 aa, Artificial Sequence
  Synthetic construct
ADDQNPWRAYLGLALLFPTDTLLLDLLWCG

SEQ ID NO: 18 - protein, 34 aa, Artificial Sequence
  Synthetic construct
ACGKEEQNPWLGAYLDLLFPLELLGLLELGLWCG

SEQ ID NO: 19 - protein, 36 aa, Artificial Sequence
  Synthetic construct
ACGLAGLAGLLGLEGLLGLPLGLLEGLWLGLELEGN

SEQ ID NO: 20 - protein, 36 aa, Artificial Sequence
  Synthetic construct
GLAGLAGLLGLEGLLGLPLGLLEGLWLGLELEGNCA

SEQ ID NO: 21 - protein, 27 aa, Artificial Sequence
  Synthetic construct
ACDDQNPWRAYLDLLFPTDTLLLDLLW

SEQ ID NO: 22 - protein, 21 aa, Artificial Sequence
  Synthetic construct
YQCKVYTGVYPFMWGGAYCFC